(19) United States
(12) Patent Application Publication
(10) Pub. No.: US 2019/0283022 A1
(43) Pub. Date: Sep. 19, 2019

(54) HIGHLY PARALLEL MICROFLUIDIC BLOOD SEPARATION DEVICE

(71) Applicants: Behrad Vahidi, Agoura Hills, CA (US); Dongjing Tang, Zhuhai (CN); Xin Ye, Los Angeles, CA (US); Xianjun Fan, Zhuhai (CN); Yanling Zhou, Zhuhai (CN)

(72) Inventors: Behrad Vahidi, Agoura Hills, CA (US); Dongjing Tang, Zhuhai (CN); Xin Ye, Los Angeles, CA (US); Xianjun Fan, Zhuhai (CN); Yanling Zhou, Zhuhai (CN)

(21) Appl. No.: 15/922,322

(22) Filed: Mar. 15, 2018

(51) Int. Cl. B01L 3/00; B03C 1/32; G01N 33/50; G01N 33/543

(52) U.S. Cl. CPC B01L 3/502715; B03C 1/32; G01N 33/5002; B01L 3/502707; B01L 2300/0893; B01L 2400/043; B01L 2300/0829; G01N 33/54326

(57) ABSTRACT

A highly parallel microfluidic blood separation device for isolation of variety of analytes for large-scale clinical trials. The device can be utilized for isolation of number of different target analytes, which may be the starting materials for variety of diagnostic methods including NGS, PCR, FISH, IHC, and others. Separation is achieved via magnetic sheath flow principals and is highly parallel. The separation device achieves effective magnetic separation, functions during sample flow in a vertical geometry and may fasten to standard multi-well plates for highly multiplexed sample recovery. Operating in vertical orientation allows multiplexing, more cards/slot/bay on instrument, allows effective use of real estate on instruments and bench tops.

### BACKGROUND

This disclosure relates to a blood separation device and in particular a device that uses sheath flow techniques in a highly parallel configuration.

Sheath flow blood separation techniques have proven effective in clinical analysis for a variety of applications. Such techniques may benefit from improvements through put for both separation operations and separated analyte collection

### SUMMARY

A highly parallel microfluidic blood separation device for isolation of variety of analytes for large-scale clinical trials. The device can be utilized for isolation of number of different target analytes, which may be the starting materials for variety of diagnostic methods including NGS, PCR, FISH, IHC, and others. Separation is achieved via magnetic sheath flow principals and is highly parallel. The separation device achieves effective magnetic separation, functions during sample flow in a vertical geometry and may fasten to standard multi-well plates for highly multiplexed sample recovery. Operating in vertical orientation allows multiplexing, more cards/slot/bay on instrument, allows effective use of real estate on instruments and bench tops.

In a first aspect, a parallel microfluidic blood separation device may be provided, including at least two parallel microfluidic channels, which include a flow region configured to direct fluid flow in a substantially vertical orientation; at least one sample reservoir (hopper) per channel in fluid communication with the flow region; at least one buffer fluid input port in fluid communication with the flow region; and, at least one fluid output port; wherein the device may be configured to accept sample fluid substantially at the top end of each channel, accept buffer fluid on the side of each channel, with pressure applied between the buffer inputs and the output port substantially at the bottom of each channel, to create sheath flow of the sample and buffer fluids in the flow region to the output port, and wherein the device is configured to be used with a separation device to trap analytes of interest from the sample fluid in the device.

In one embodiment of the first aspect, there may be at least 8 parallel channels in the device arranged linearly and with spacing of the outlet ports compatible with standard well plate reservoir separation dimensions. In another embodiment of the first aspect, there may be at least one of 16 or 24 parallel channels in the device arranged linearly and with spacing of the outlet ports compatible with standard well plate reservoir separation dimensions. In one embodiment of the first aspect, the separation device may be magnetic, configured to attract magnetically tagged analytes to a sidewall of each channel and hold the analytes in place to remain in the device after the flow operations are complete.

In another embodiment of the first aspect, the device may be made of at least two plastic parts, one part injection molded with the hopper, input ports, output ports and flow region structures, and the other part a cover piece adhered to the injection molded part to complete the device. In one embodiment of the first aspect, at least one flexible gasket material may be injected molded to at least one of the ports. In another embodiment of the first aspect, the flexible gasket may be on the output port, which is disposed substantially on the bottom of each channel, and the gasket is configured to seal to a well plate reservoir.

In another embodiment of the first aspect, multiple separation devices with separated analyte may be stacked together in a holding fixture and sealed to multiple rows of well plate reservoirs. In one embodiment of the first aspect, a spin fixture may be configured to spin multiple stacks of separation devices sealed to well plates together for highly parallel sample extraction.

In one embodiment of the first aspect, there may be two side buffer fluid inputs, on opposite sides of the flow region on each channel configured to sheath the sample flow on two sides. In another embodiment of the first aspect, the device may be configured to, after flow separation is complete, to interface each channel's output port to a collection device wherein the separation device and the collection device may be spun to draw the analyte into the collection device. In one embodiment of the first aspect, the collection device may be a well plate and the separation device output ports are sealed each channel to a row of well plate reservoirs.

In a second aspect, a method for highly parallel microfluidic blood separation may be provided using a separation device including at least two parallel microfluidic channels, comprising a flow region configured to direct fluid flow in a substantially vertical orientation; including the steps of filling a sample hopper per channel which is in fluid communication with the flow region; adding buffer fluid to an input port in each channel in fluid communication with the flow region; and, extracting buffer and sample fluid from an output port; wherein sample fluid may be accepted substantially at the top end of each channel, buffer fluid is accepted on the side of each channel, with pressure applied between the buffer inputs and the output port substantially at the bottom of each channel, to create sheath flow of the sample and buffer fluids in the flow region to the output port, and wherein a separation device is used to trap analytes of interest from the sample fluid in the device.

In one embodiment of the second aspect, there may be at least 8 parallel channels in the device arranged linearly and with spacing of the outlet ports compatible with standard well plate reservoir separation dimensions. In another embodiment of the second aspect, there may be at least one of 16 or 24 parallel channels in the device arranged linearly and with spacing of the outlet ports compatible with standard well plate reservoir separation dimensions.

In one embodiment of the second aspect, the separation device may be magnetic which attracts magnetically tagged analytes to a sidewall of each channel and holds the analytes in place to remain in the device after the flow operations are complete. In another embodiment of the second aspect, there may be two side buffer fluid inputs; on opposite sides of the flow region on each channel and the buffer fluid sheathes the sample flow on two sides.

In one embodiment of the second aspect, the method may further include the steps of after flow separation is complete, interfacing each channel's output port to a collection device wherein the separation device and the collection device are spun to draw the analyte into the collection device. In another embodiment of the second aspect, the collection device may be a well plate and the separation device output ports are sealed each channel to a row of well plate reservoirs. In one embodiment of the second aspect, multiple separation devices with separated analyte may be stacked together in a holding fixture and sealed to multiple rows of well plate reservoirs.

### BRIEF DESCRIPTION OF THE DRAWINGS

Aspects and advantages of the embodiments provided herein are described with reference to the following detailed description in conjunction with the accompanying drawings. Throughout the drawings, reference numbers may be re-used to indicate correspondence between referenced elements. The drawings are provided to illustrate example embodiments described herein and are not intended to limit the scope of the disclosure.

FIGS. 1A and 1B show an exemplary embodiment of one channel of a vertical highly parallel blood separation device;

FIGS. 2A and 2B 1B show an exemplary embodiment of a vertical highly parallel blood separation device;

FIG. 3 shows an construction details of an exemplary embodiment of a vertical highly parallel blood separation device;

FIG. 4 shows compatibility between an exemplary embodiment of a vertical highly parallel blood separation device with standard well plates;

FIG. 5 shows an exemplary embodiment of devices to hold and spin a large number of vertical highly parallel blood separation devices for separated analyte offload offload;

FIG. 6 depicts sealing between a vertical highly parallel blood separation device and a well plate for sample analyte offload;

### DETAILED DESCRIPTION OF ILLUSTRATIVE EMBODIMENTS

The current disclosure is directed to a highly parallel blood separation device that is configured to operate with vertical flow, contain a plurality of parallel flow channels and be compatible with standard sample analysis devices such as well plates.

The current disclosure is based on a sheath flow blood separation device described in U.S. Pat. No. 8,263,287 entitled “Sheath Flow Devices and Methods”, issued Sep. 11, 2012 and commonly owned by the owners of the current application. U.S. Pat. No. 8,263,287 is incorporated by reference in its entirety. U.S. Pat. No. 8,263,287 describes the techniques of a sample fluid flow sheathed by one or more buffer fluid flows. Analytes of interest, such as cancer cells in blood, are tagged in some fashion, such as bonding to magnetic particles. When exposed to a separation mechanism, such as a magnetic field, analytes of interest are separated from the sample flow into one of the buffer flows and trapped within the sheath flow device. Once flow is complete, pellets of analyte remain in the device and may be removed for analysis. One technique for removal is a spin elution technique described in U.S. Provisional Application 61/702,730 entitled “Spin Elute Tube” filed Sep. 18, 2012, since published as International Publication WO2014/046942 A1. This application is also commonly owned by the owners of the current application and is incorporated in its entirety by reference.

The current application builds on the foundation of the incorporated references but further discloses key beneficial features that achieve significant performance benefits.

In some embodiments smaller sample volumes at higher sample density may be provided. Advantageously these features allow for much faster assay times.

In some embodiments sample fluids may be end loaded and end exhausted. Advantageously this arrangement requires no redirection of sample flow leading to reduced complexity and cost of manufacturing.

In some embodiments the collection of separated analyte of interest is compatible with standard multi sample analysis such as well plates. Advantageously this arrangement allows for direct sample delivery to high throughput analysis devices.

In diagnostics, commercial scalability is only possible if and when economics of the test are favorable, and the capability of processing large numbers of clinical samples exists. The current disclosure targets a selected set of applications and caters to population-scale testing with improved performance compared to current systems.

With existing systems, as amounts of testing materials/analytes lessen, sample loss (losing cells/analytes of interest) rapidly becomes more of an issue. This current disclosure also introduces a direct transfer method for minimal sample loss into standard receptacles design (i.e. PCR tubes, multi-well plates, & etc. . . . ).

The current disclosure provides a highly parallel microfluidic blood separation device for isolation of variety of analytes for large scale clinical trials. The device can be utilized for isolation of number of different target analytes such as cfDNA, CTCs, RNA, and Exsosomes, which are the starting materials for variety of diagnostic methods including NGS, PCR, FISH, IHC, and others. Separation is achieved via the magnetic sheath flow principals of the incorporated references but enhanced to be highly parallel. The device of the current disclosure provides enhances magnetic separation, functions during sample flow in a vertical geometry and fastens to standard multi-well plates for highly multiplexed sample recovery. Vertical orientation of the system eliminates the need for a end buffer, thereby reducing cost and improving automation compatibility. Operating in vertical orientation allows multiplexing, more cards/slot/bay on instrument, allows maximum use of real estate on instruments and bench tops. The current separation device is optimized for detection performance, low cost/testing, and ease of manufacturing, and provides an elegant and effective way to collect analyte pellets.

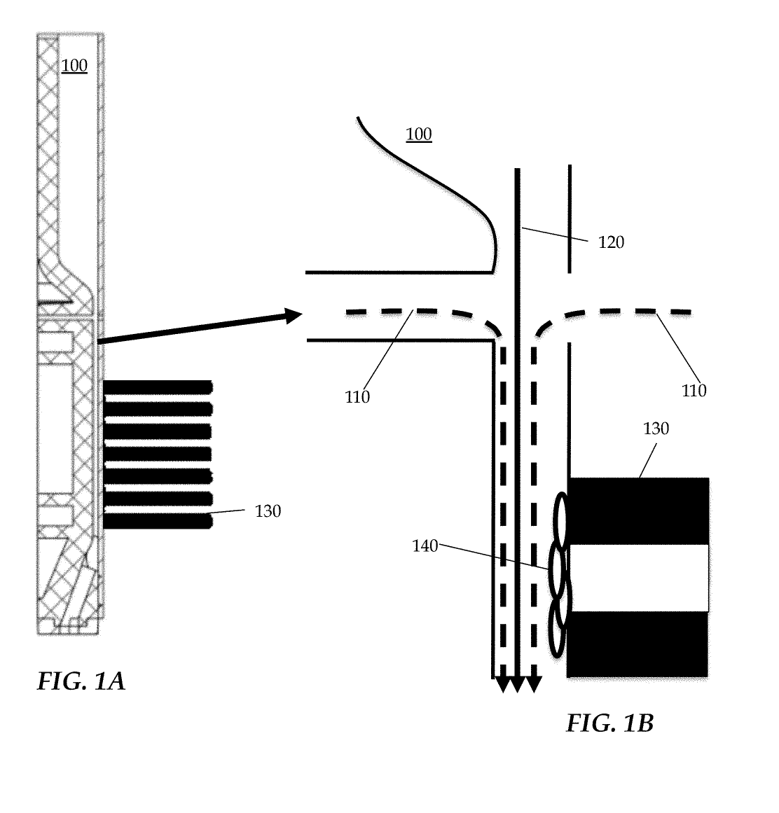

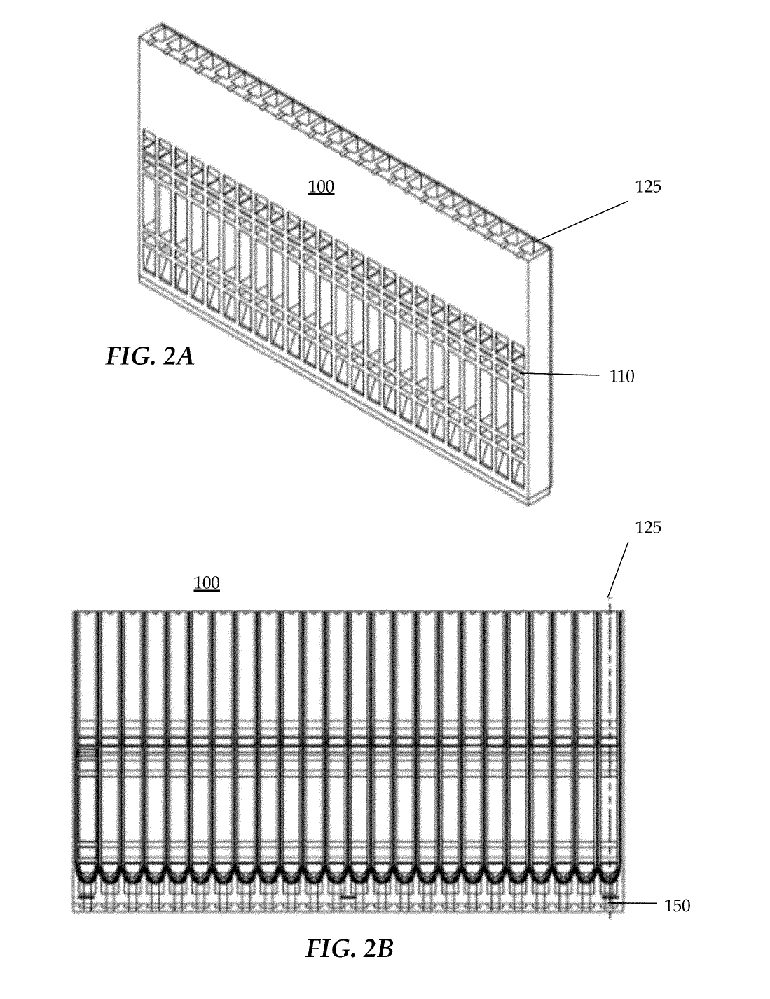

Referring to FIGS. 1A, 1B, 2A, and 2B, several views are shown of a cross-section of one channel of the current separation device 100, Sample and buffer flow features and the overall configuration of an exemplary multi-channel blood separation device. There are 2 side inlet ports 110 per each channel and a single outlet port 150 on the bottom services each flow region 120 for each channel. Top portion of each channel 125 is open to atmosphere and functions as sample reservoir. Sample reservoirs (hopper) are an integral part of the device (minimizes sample loss, simplifies liquid handling). Similar to FACS technology, where there have been major innovation/developments in last decade, sample containing target analytes, the solid arrow, gets hydrodynamically entrained/focused between 2 streams of inert fluid, the dashed arrows, injected (via positive pressure) through the side inlets.

As can be seen in FIGS. 2A and 2B, the device 100 in the embodiment shown is a linear array of channels. The center-to-center distance may be chosen to match the center to center well separation for standard well plates. For example for a 480 wellplate the channel-to-channel center separation at the bottom would 0.18 inches. For the 480 well plate compatible embodiment, example dimensions might be a thickness of the device of −0.3 inches or less and the total length for a 24 channel device would be ˜4.3 inches with height of each channel ˜2.4 inches. Common well plate sizes include arrays of wells with 8, 16 or 24 wells, as well as other sizes, along one dimension. The number of channels and the channel-to-channel separation may be chosen to be compatible with any of these standards or other collection devices.

An example workflow using magnetic separation for an exemplary device embodiment starts with blood/other bodily fluids arriving at a lab in their standard and respective receptacles. The sample then goes through a preparation process where the cells/analytes of interest are labeled with target specific antibody decorated ferromagnetic beads. Individual samples are subsequently run through the current device 100, either in parallel or successively in any desired order. As the sample passes through the sheath flow region of the device 120, e.g. the region between the side buffer inputs and the bottom end outlet port of the device, an external magnetic filed generator 130 attracts only the target cells/analytes 140 bound to magnetic beads. The beads are previously conjugated to specific antibodies that complement a specific antigen on cells/analytes of interest. Non-specific absorption of non-target cells/analytes is minimized, resulting in a highly purified population of target cell/analytes 140 due to the sheath flow effect described above. The buffer and sample fluids exit each channel out the bottom outlet 125.

Flow rates pressures over an analyte run cycle, flow rates are 3-15 mL/h driven by the difference between the two positive pressure side flows & suction (−pressure flow) from the outlet port. is analyte's flow rate. Once the sample is thru, there are series of rinses that happen at higher flow rates. Pressures are small as well. On the order of 0-600 Pa. Following the automated rinses on the instrument, a small pellet of selected population of cells/analytes 140 remains in the device 100.

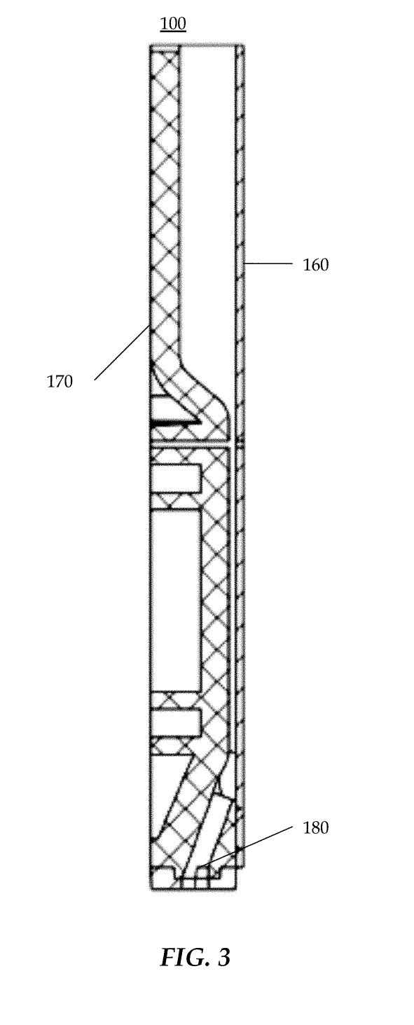

Referring to FIG. 3 construction details of an exemplary embodiment of the current device will be discussed. Use is made of injection/injection-compression molding, hot embossing, and casting techniques which are both scalable and economical.

All complex/intricate parts of the device 100, such as the hopper, inlet and outlet ports and flow region are allocated to one side of/half of the device 170 that is injection molded This includes ribs on the backside of the flow region of the device 100 behaving as energy directors for good weld and precision for indexing the device 100 onto external devices such as microfluidic systems, well plates or analytical instruments. A more flexible plastic material 180 may be injected through the mold that produces the gaskets on the base of the card around the outlet port, or in the vicinity of other ports as well. One important element about the routing of the device 100 is the avoidance of chemical contamination. The sample should not be contaminated with debris/contaminants from outside and instrument and its components are not contaminated by the analyte. The biological sample only comes in contact with the gasket therefore the gasket being part of the device, which may be disposable after use. The gasket 180 on the outlet of device 100 may also be seamless (Cells don't get trapped/left behind) and integral to the device 100. Over-molded from a more flexible material it may provide a hermetic seal with instrumentation used for sample delivery and separation while in operation and with multi-well plates for sample recovery after processing. Final step is ultrasonically welding flat cover sheet of plastic 160 to the structural side of the device 170.

Materials for some embodiments of the device may be Polystyrene, the same as used for many multi-well plates. Polycarbonate could also be used, as Polystyrens and Polycarbonate have the same shrink rate in injection molding. A suitable gasket material is KRATON, 32A SHORE, but there are many more options suitable flexible materials. Process is called 2-shot molding/over molding, where PS is shot into the mold, cured followed by 2^{nd} shot of diff material. It makes a Seem-less connection, while providing great accuracy/tolerance for attachment to the plates. In one embodiment, the cover plate thickness is 0.02″, i.e. distance between the magnet side and the captured analyte cells, the sample hopper volume is ˜300 uL/Channel, and the side ports are rectangular and connect to fluidics via distribution manifolds.

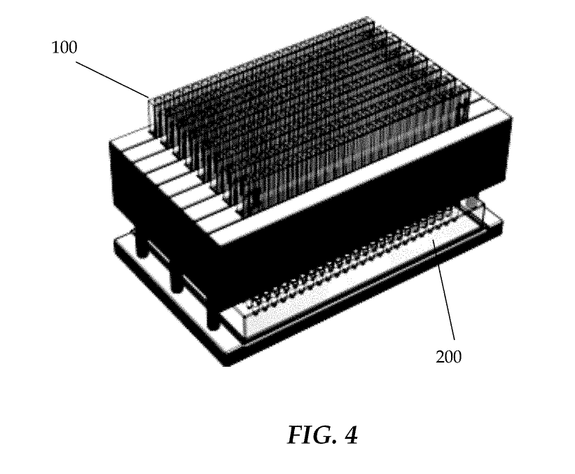

Once the channels of a device have been processed for separation, each processed channel will have a pellet of target analyte remaining in the flow region, which needs to be offloaded. Processed sample off-load in some embodiments may involve fastening to standard multi-well plates via an adaptor. As shown in FIG. 4, device 100 may correspond and mate to one row of a well plate 200. Every other row on the plate will be occupied by a caddy/adaptor. For example, for 24-channel device as shown, one could assemble 8 cards to a 384 well plate (16×24). That is 192 samples per plate/centrifuge bucket.

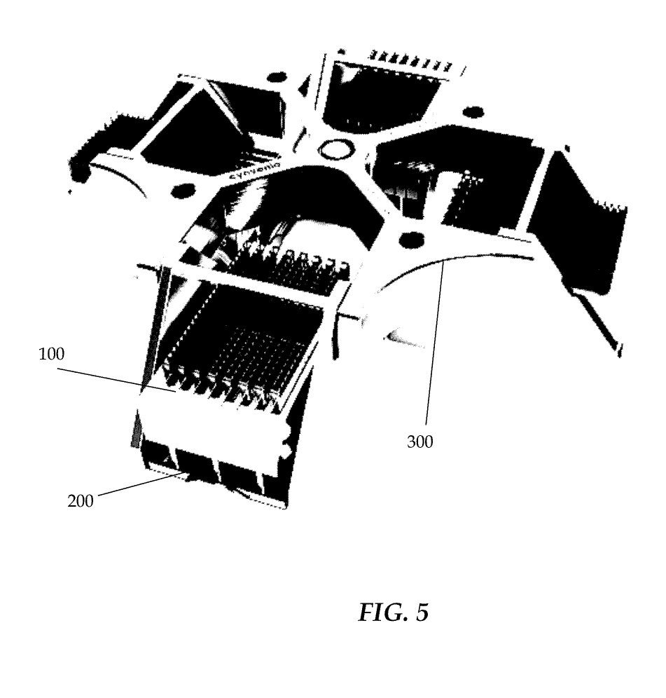

In one embodiment, the process may be highly multiplexed and allow fast and highly parallel recovery of cells/analytes of interest from microfluidic device into a standard well plate. As shown in FIG. 5. Devices 100 may be bundled into a carrier designed to space the linear devices from each other at the center-to-center well distance for a well plate 200. As shown in the Figure, many of the well plate's reservoirs may be filled at once from a suitable bundle of processed devices 100. This may vary from a portion of the well plate reservoirs, with some of the rows used as attachment areas, up to an including all of the well plate reservoirs. However any number of devices from one to as many as would cover an entire well plate number could be used. Also shown in FIG. 5 is multi-bindle spin elution fixture 300, which could elute processed analyte into multiple entire well plates simultaneously. Despite slow flow rates per channel each card processes 8/16/24 samples in less than 5 min. Different fluidic manifolds may be used. Manifolds for an 8-channel device, for example, may split lines from 1 to 8. This is because analyte is concentrated to ˜300 uL (hopper volume/channel

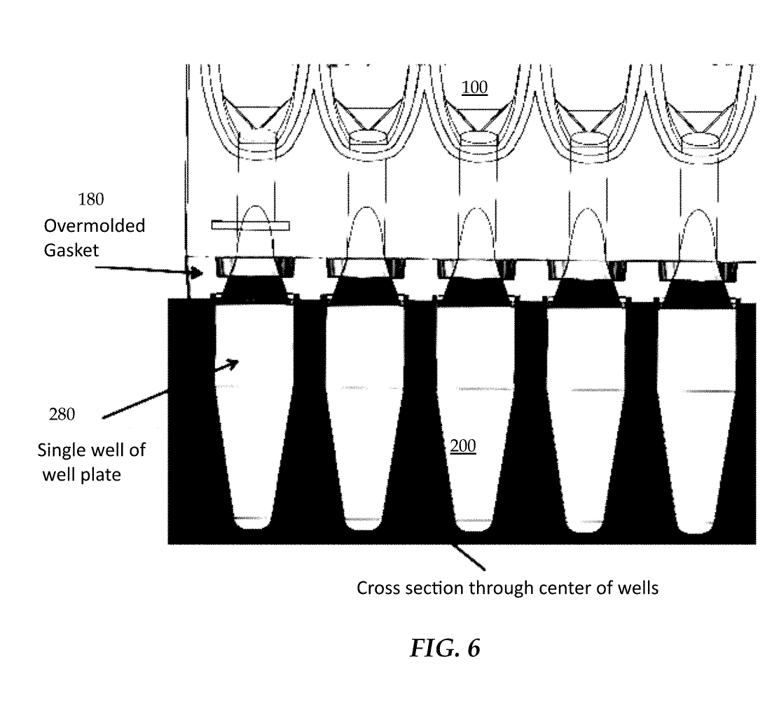

FIG. 6 shows details of device 100 output ports mated to well plate 280 reservoirs. Internal volumes/channels of ˜30 uL are to meet the requirement s for mating with the well plates, with no overflow of wells. The lips of individual over molded gaskets 180 protrude into interior wall of well, and the well plate lip presses into gasket. Small channels in the gasket allow air trapped in the wells. As the air escapes the well during spin elution of the device such as with the spine elution fixture of FIG. 5, centrifugal force pushes the pellet of target moieties into the wells.

The embodiments described herein are exemplary. Modifications, rearrangements, substitute materials, alternative elements, etc. may be made to these embodiments and still be encompassed within the teachings set forth herein.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices and components illustrated can be made without departing from the spirit of the disclosure. As will be recognized, certain embodiments described herein can be embodied within a form that does not provide all of the features and benefits set forth herein, as some features can be used or practiced separately from others. All changes, which come within the meaning and range of equivalency of the claims, are to be embraced within their scope.

1. A parallel microfluidic blood separation device comprising:
at least two parallel microfluidic channels, comprising a flow region configured to direct fluid flow in a substantially vertical orientation;
at least one sample hopper per channel in fluid communication with the flow region;
at least one buffer fluid input port in fluid communication with the flow region; and,
at least one fluid output port; wherein
the device is configured to accept sample fluid substantially at the top end of each channel, accept buffer fluid on the side of each channel, with pressure applied between buffer inputs and the output port substantially at the bottom of each channel, to create sheath flow of the sample and buffer fluids in the flow region to the output port, and wherein the device is configured to be used with a separation device to trap analytes of interest from the sample fluid in the device.

2. The device of claim 1 wherein the there are at least 8 parallel channels in the device arranged linearly and with spacing of the outlet ports compatible with standard well plate reservoir separation dimensions.

3. The device of claim 2 wherein their are at least one of 16 or 24 parallel channels in the device arranged linearly and with spacing of the outlet ports compatible with standard well plate reservoir separation dimensions.

4. The device of claim 2 wherein the separation device is magnetic, configured to attract magnetically tagged analytes to a sidewall of each channel and hold the analytes in place to remain in the device after the flow operations are complete

5. The device of claim 1 wherein the device is made of at least two plastic parts, one part injection molded with the hopper, input ports, output ports and flow region structures, and the other part a cover piece adhered to the injection molded part to complete the device.

6. The device of claim 5 wherein at least one flexible gasket material is injected molded to at least one of the ports.

7. The device of claim 5 wherein the flexible gasket is on the output port, which is disposed substantially on the bottom of each channel, and the gasket is configured to seal to a well plate reservoir.

8. The device of claim 1 wherein there are two side buffer fluid inputs, on opposite sides of the flow region on each channel configured to sheath the sample flow on two sides.

9. The device of claim 1 configured to, after flow separation is complete, to interface each channel's output port to a collection device wherein the separation device and the collection device are spun to draw the analyte into the collection device.

10. The device of claim 9 wherein the collection device is a well plate and the separation device output ports are sealed each channel to a row of well plate reservoirs.

11. The device of claim 10 wherein multiple separation devices with separated analyte are stacked together in a holding fixture and sealed to multiple rows of well plate reservoirs.

12. The device of claim 11 wherein a spin fixture is configured to spin multiple stacks of separation devices sealed to well plates together for highly parallel sample extraction.

13. A method for highly parallel microfluidic blood separation using a separation device comprising at least two parallel microfluidic channels, comprising a flow region configured to direct fluid flow in a substantially vertical orientation, comprising:
Filling a sample hopper per channel which is in fluid communication with the flow region;
Adding buffer fluid to an input port in each channel in fluid communication with the flow region; and,
Extracting buffer and sample fluid from an output port; wherein
sample fluid is accepted substantially at the top end of each channel, buffer fluid is accepted on the side of each channel, with pressure applied between the buffer inputs and the output port substantially at the bottom of each channel, to create sheath flow of the sample and buffer fluids in the flow region to the output port, and wherein a separation device is used to trap analytes of interest from the sample fluid in the device.

14. The method of claim 13 wherein there are at least 8 parallel channels in the device arranged linearly and with spacing of the outlet ports compatible with standard well plate reservoir separation dimensions.

15. The method of claim 14 wherein their are at least one of 16 or 24 parallel channels in the device arranged linearly and with spacing of the outlet ports compatible with standard well plate reservoir separation dimensions.

16. The method of claim 13 wherein the separation device is magnetic which attracts magnetically tagged analytes to a sidewall of each channel and holds the analytes in place to remain in the device after the flow operations are complete

17. The method of claim 13 wherein there are two side buffer fluid inputs, on opposite sides of the flow region on each channel and the buffer fluid sheathes the sample flow on two sides.

18. The method of claim 13 further comprising, after flow separation is complete, interfacing each channel's output port to a collection device wherein the separation device and the collection device are spun to draw the analyte into the collection device.

19. The method of claim 13 wherein the collection device is a well plate and the separation device output ports are sealed each channel to a row of well plate reservoirs.

20. The method of claim 19 wherein multiple separation devices with separated analyte are stacked together in a holding fixture and sealed to multiple rows of well plate reservoirs.
